(19) United States
(12) Patent Application Publication
(10) Pub. No.: US 2005/0113452 A1
(43) Pub. Date: May 26, 2005

(54) Composition and dosage form for sustained effect of levodopa

(76) Inventors: Moshe Flashner-Barak, Petach Tikva (IL); E. Itzhak Lerner, Petach Tikva (IL); Vered Rosenberger, Jerusalem (IL)

Correspondence Address: KENYON & KENYON, ONE BROADWAY, NEW YORK, NY, 10004, US

(21) Appl. No.: 10/966,090

(22) Filed: Oct. 14, 2004

Related U.S. Application Data
(60) Provisional application No. 60/512,973, filed on Oct. 20, 2003.

(51) Int. Cl. A61K 31/445; A61K 31/198; A61K 31/137

(52) U.S. Cl. 514/567; 514/317; 514/649

(57) ABSTRACT

The present invention encompasses compositions for the treatment of Parkinson's disease comprising a therapeutically effective amount of levodopa or a metabolic precursor thereof and at least one dopamine transport inhibitor in sufficient amount to decrease dopamine degradation, wherein the dopamine transport inhibitor is administered to avoid dyskinesia.

### CROSS-REFERENCE TO RELATED APPLICATIONS

This application claims the benefit of provisional application Ser. No. 60/512,973, filed Oct. 20, 2003, which is incorporated herein by reference in its entirety.

### FIELD OF THE INVENTION

The present invention is directed to immediate release and sustained release delivery formulations of levodopa wherein dopamine transport is inhibited to prolong dopamine presence in the brain. More particularly, the invention is directed to levodopa compositions and dosage forms that release levodopa in an immediate or sustained release manner and extend the effects of dopamine using a dopamine transport inhibitor.

### BACKGROUND OF THE INVENTION

Parkinson's Disease is a degenerative condition associated with reduced dopamine concentrations in the basal ganglia region of the brain. The deficiency is considered to be caused by oxidative degradation of dopaminergic neurons in the substantia nigra. The preferred course of therapy is to restore dopamine concentration in the brain by administration of levodopa, a metabolic precursor of dopamine that, unlike dopamine, is able to cross the blood-brain barrier. The metabolic transformation of levodopa to dopamine is catalyzed by the aromatic L-amino acid decarboxylase enzyme. This enzyme is found throughout the body including gastric juices and the mucosa of the intestine. Treatment with levodopa alone requires administration of large doses of the drug due to extracerebral metabolism by this enzyme. The resulting high concentration of extracerebral dopamine causes nausea in some patients. To overcome this problem levodopa is usually administered with an inhibitor of the aromatic L-amino decarboxylase enzyme such as carbidopa.

Levodopa eases the symptoms of Parkinsonism by temporarily boosting dopamine concentration in the central nervous system, but it is not a cure. During prolonged treatment of the disease with levodopa, the body typically becomes less sensitive to levodopa concentration in the brain. The body requires more frequent dosing to suppress the manifestations of the disease: tremor, muscular rigidity, lack of facial expression, and altered gait. As the blood plasma concentration drops, the return of disease manifestations in the so-called “off state,” signals the need for immediate administration of another dose. There is, unfortunately, a delay between ingestion of levodopa and a return to the “on state” suppression of the disease symptoms. Aggressive administration of levodopa to circumvent off state symptoms of rigidity and akinesia, can lead to equally disabling involuntary motions called dyskinesias.

From the foregoing, it will be appreciated that it would be highly desirable to administer levodopa as a sustained release oral dosage form capable of stabilizing the serum level of levodopa in a patient. Levodopa/carbidopa is currently available in SINEMET® CR controlled release tablets (DuPont Pharma) that slowly erode to release the actives. According to the Physician's Desk Reference, 54th ed., the tablets use a polymeric based drug delivery system. Prolonged suppression of disease manifestations with these tablets is limited by the mechanism of absorption of levodopa from the gastrointestinal tract. Levodopa is absorbed by the active transport mechanism for amino acids, which is most active in the duodenum region of the small intestine. Sustained release is therefore limited by the transit time of the dosage form through the stomach and duodenum which, though highly variable from individual-to-individual and dependent upon nutritional state, typically takes only about 3 to 4 hours. Levodopa released after the 3-4 hour therapeutic window has passed is not bioavailable. SINEMET® CR carbidopa-levodopa controlled release tablets have about 75% of the bioavailability of SINEMET® carbidopa-levodopa conventional release tablets. Physicians Desk Reference, p. 979, (54th edition, Medical Economics Co., publisher, 2000).

Therefore, there is a need for a controlled release levodopa oral dosage form that is able to deliver levodopa to a patient's bloodstream over a longer time period than is currently possible without resort to a regimen of frequent dosing, and the fluctuations in plasma levodopa levels that occur with frequent dosing. Further, there is a need for improvement in controlled-release forms that improves the bioavailability of levodopa as well as lowers the dosage frequency.

Another problem that could be addressed with an improved controlled release levodopa delivery vehicle is the reduction in plasma levodopa concentration that occurs while a Parkinson's patient is sleeping. Parkinson's patients usually awaken in the morning in the off state and must wait for a morning dose of levodopa to take effect before they can function comfortably. It would be highly desirable to be able to take levodopa in the evening, while under the therapeutic effect of a previous dose, and wake up in the morning without the manifestations of the disease. For such purpose, the drug delivery vehicle ideally would not only extend the release of levodopa over time, but would also delay release of levodopa until the early morning hours before the patient awakens so that the patient would awaken when the therapeutic effect of the dose is near its maximum.

Many conventional delayed release dosage forms possess a coating that dissolves slowly in gastrointestinal fluid. Release of the active is delayed until dissolution of the coating allows gastrointestinal fluid to contact a core of the dosage form containing the drug. However, coatings alone are unsuitable for delaying release of levodopa because of the site specificity of levodopa absorption. Delayed release vehicles that are not retained in the stomach during the period before onset of release will have passed through the duodenum, missing the window of bioavailability.

As reviewed by Hwang, Park and Park in “Gastric Retentive Drug-Delivery Systems,” Critical Reviews in Therapeutic Drug Carrier Systems, 1998, 15, 243-284, a variety of approaches have been attempted for controlling the gastric retention time of pharmaceuticals by altering their size, shape, density and surface properties.

Levodopa is not amenable to long term slow release dosage forms that progress through the gastrointestinal tract. Dosage forms retained in the stomach for many hours are a possible solution to this problem and have been developed (see U.S. patent application Ser. No. 09/887,204) but have not been commercialized to date. Currently, large peaks of levodopa concentrations are necessary to be delivered in order to give a longer period wherein enough drug remains in the brain to be effective despite the short half life. The large peaks and the short time until the troughs due to the short half life cause large fluctuations from dyskinesia to akinesia both of which are disabling.

Another approach to achieving a constant dopamine level in the brain is to work at the level of brain biochemistry. Dopamine in the brain, whether released by a pre-synaptic neuron or supplied by the delivery of levodopa through the brain blood barrier, is removed from the junction by mechanisms of dopamine uptake to stop information transfer. Partial blocking of the dopamine uptake could result in more constant dopamine levels in the brain without the need to modify the levodopa profile in the blood. Methylphenidate, a relatively safe drug used to treat children suffering from Attention Deficit Disorder (ADD) or Attention Deficit Hyperactivity Disorder (ADHD), is a dopamine transport inhibitor. Methylphenidate has been used with levodopa in Parkinson's disease patients, however, only to cause severe dyskinesia and other motor effects of levodopa on the patients, especially when the two drugs are delivered together. Camicioli, et al. “Methylphenidate Increases the Motor Effects of L-Dopa in Parkinson's Disease: a Pilot Study,” Clin. Neuropharmacol. 24(4), 208-213 (2001).

The present invention uses a formulation to achieve sustained release and/or delayed release of levodopa to a patient suffering from a central nervous system dopamine deficiency disease and to prolong the effects of levodopa which overcomes the severe dyskinesia induced by prior formulations.

### SUMMARY OF THE INVENTION

The present invention encompasses a composition for the treatment of Parkinson's disease comprising a therapeutically effective amount of levodopa or a metabolic precursor thereof; and at least one dopamine transport inhibitor in sufficient amount to decrease dopamine removal from the brain without causing dyskinesia or other undesirable motor effects. In one embodiment, the composition is adapted to release the dopamine transport inhibitor in a manner such as to avoid dyskinesia. In another embodiment, the dopamine transport inhibitor is methylphenidate, present in an amount of about 3 mg to about 60 mg. In another embodiment of the invention, the levodopa or metabolic precursor thereof is present in an amount of about 50 mg to about 300 mg.

The composition comprising a therapeutically effective amount of levodopa or a metabolic precursor thereof; and at least one dopamine transport inhibitor may further comprise at least one carboxylase enzyme inhibitor. The carboxylase enzyme inhibitor may be at least one of carbidopa or benserazide. The carboxylase enzyme inhibitor may be present in an amount of about 10 mg to about 100 mg.

The present invention also encompasses a pharmaceutical composition for treating, preventing, or ameliorating Parkinson's disease comprising an immediate release or sustained release delivery formulation of levodopa or a metabolic precursor thereof; and a formulation of at least one dopamine transport inhibitor wherein the dopamine transport inhibitor is released immediately after a delay about 2 hours to about 7 hours.

In one embodiment of the invention, the pharmaceutical composition of an immediate release or sustained release delivery formulation of levodopa or a metabolic precursor thereof; and a formulation of at least one dopamine transport inhibitor may further comprise at least one decarboxylase enzyme inhibitor in an immediate release formulation or in a sustained release delivery formulation. Typically, the sustained release formulation is released over about 1 hour to about 4 hours. The pharmaceutical composition may be shaped into a bilayer tablet or a sheathed tablet.

The present invention also encompasses a pharmaceutical composition for treating, preventing, or ameliorating Parkinson's disease comprising an immediate release or sustained release delivery formulation of levodopa or a metabolic precursor thereof; and a sustained release delivery formulation of at least one dopamine transport inhibitor wherein the dopamine transport inhibitor is released over a period of time of about 1 hour to about 6 hours after a delay of about 2 hour to about 7 hours. In one embodiment of the invention, the pharmaceutical composition comprising an immediate release or sustained release delivery formulation of levodopa or a metabolic precursor thereof; and a sustained release delivery formulation of at least one dopamine transport inhibitor may further comprise a decarboxylase enzyme inhibitor in an immediate release or in a sustained release delivery formulation, wherein the sustained release delivery formulation is released over about 1 hour to about 4 hours.

Another embodiment of the invention encompasses a method of treating Parkinson's disease comprising administering a therapeutically effective amount of levodopa or a metabolic precursor thereof; and at least one dopamine transport inhibitor in sufficient amount to decrease dopamine elimination, wherein the dopamine transport inhibitor is administered to avoid dyskinesia. In another embodiment, the dopamine transport inhibitor may be methylphenidate and may be administered in an amount an amount of about 3 mg to about 60 mg. In one embodiment, the levodopa or metabolic precursor thereof may be administered in an amount of about 50 mg to about 300 mg. The method of treating Parkinson's disease may also comprise administering at least one carboxylase enzyme inhibitor, wherein the carboxylase enzyme inhibitor may be carbidopa, benserazide, or a combination thereof. In another embodiment of the invention, the carboxylase enzyme inhibitor may be administered in an amount of about 10 mg to about 100 mg.

### BRIEF DESCRIPTION OF THE FIGURES

FIG. 1 illustrates the cumulative release of methylphenidate, levodopa, and carbidopa in sustained release delivery formulation.

FIG. 2 illustrates the release profile of levodopa as an average plasma concentration of levodopa over time in a treatment using an embodiment of the invention as compared to a reference.

FIG. 3 illustrates the release profile of carbidopa as an average plasma concentration of carbidopa over time in a treatment using an embodiment of the invention as compared to a reference.

FIG. 4 illustrates the release profile of methylphenidate as an average plasma concentration of methylphenidate over time in a treatment using an embodiment of the invention as compared to a reference.

FIG. 5 illustrates the release profile of ritanilic acid as an average plasma concentration of ritanilic acid over time in a treatment using an embodiment of the invention as compared to a reference.

### DETAILED DESCRIPTION OF THE INVENTION

To improve the treatment of Parkinson's Disease when using levodopa as a source of dopamine in the brain, it is necessary to find a method of providing brain levels of dopamine wherein the levels are constant. Optimally, the drug concentration troughs are shallower and the peaks are lower than obtained by currently used therapies. Dopamine brain levels follow levodopa blood levels, because levodopa in the blood is the main source of dopamine in the brain, in particular for patients suffering from Parkinson's disease. Once levodopa travels through the blood brain barrier, levodopa is bioconverted into dopamine within the brain. Methods to attain constant levodopa concentrations include controlling the rate of delivery of dopamine or controlling the rate of accrual of dopamine in the brain. However, maintaining nearly constant levels of levodopa in the blood has proven to be an elusive goal due to special characteristics of levodopa.

To obtain extended duration of drug levels in the blood, drugs with long elimination half-lives are preferred. Levodopa, however, has a short half-life of about one and a half hours in the presence of carbidopa and shorter without. Residence time of a dosage form in the duodenum is short and is measured in minutes and not hours. Levodopa is absorbed when released in the stomach and by an active transport mechanism in the duodenum. Drugs that absorb throughout the length of the small intestine, but not in the colon, can be designed to deliver a zero order slow release profile over about five hours, i.e. the time of small intestine transit (three to four hours) plus the time the drug resides in the stomach (if drug release starts in the stomach). For drugs absorbed in the small intestine and colon, zero order release profiles of 12 hours or more are desired. Any slow release form that releases dopamine after the sum total of residence time in the stomach and duodenum wastes the amount of drug released after passage through the duodenum, thereby lowering bioavailability.

When administered with a proper drug delivery regimen, we have found that the detrimental results associated with the co-delivery of levodopa and at least one dopamine transport inhibitor can be avoided and beneficial results achieved. Delivery of dopamine transport inhibitors too early in the levodopa-in-blood release profile enhances the adverse motor effects caused by high levels of dopamine in the brain. However, by properly adjusting the levodopa dose and proper timing of the dopamine transport inhibitor to block the dopamine transporter, levodopa therapy leading to constant levels of dopamine in the brain can be achieved. Preferably, the dopamine transport inhibitor is administered as the dopamine levels start to decrease.

During normal brain function dopamine is released in the synapse by neuron cells and eliminated by transport proteins. Co-treatment with a drug that inhibits the transport protein allows the dopamine to reside longer in the brain, thereby making the effective drug troughs shallow. Additionally, the efficient use of the method lowers concentration peaks by lowering levodopa dosing levels. Proper timing of the co-treatment with the two drugs is essential. Administering the dopamine transport inhibitor too early in the levodopa-in-blood profile will result in peaks of brain dopamine and problems of dyskinesia. Administering the dopamine transport inhibitor too late in the levodopa-in-blood profile will result in to little advantage because the dopamine levels in the brain will have been already depleted by the normal elimination processes. Proper timing of the transport inhibitor delivery to slightly after the predicted peak in brain dopamine concentration, and keeping the inhibitor in place for some time, extends the time that effective concentrations of the dopamine are present in the brain. However, extending the time of the transport inhibition too long can have deleterious effects since it may lead to too high a dopamine concentration upon the next dosing of the levodopa.

The present invention provides a sustained release pharmaceutical composition that releases levodopa upstream of the duodenum over a period of hours by delaying the release of levodopa and/or delaying the removal of levodopa from the synapse. The invention further provides dosage forms comprising levodopa and at least one dopamine transport inhibitor. The administration of the dopamine transport inhibitor may be delayed such that release coincides with the time the dopamine concentration level starts to decrease. The formulation may optionally include a decarboxylase enzyme inhibitor. A levodopa metabolic precursor like the levodopa ethyl ester of U.S. Pat. No. 5,840,756 may be substituted for levodopa in the various embodiments of the invention. U.S. Pat. No. 5,840,756 is hereby incorporated by reference. Typically, levodopa is present in an amount from about 50 mg to about 300 mg, preferably from about 100 mg to about 200 mg and more preferably, levodopa is present in an amount of about 100 mg to about 150 mg per dose.

The dopamine transport inhibitor is a compound capable of delaying the dopamine transporter from removing dopamine from the brain. In other words, the dopamine transport inhibitor precludes or diminishes the removal rate of dopamine by the dopamine transporter, thereby prolonging a concentration of dopamine in the brain. Dopamine transporter inhibitors include, but are not limited to, methylphenidate. In the formulation of the invention, methylphenidate may be present in an amount about 1 mg to about 60 mg, preferably from 1 mg to about 15 mg, more preferably, from about 5 mg to about 10 mg, and most preferably methylphenidate may be present in an amount of about 10 mg per dose.

Optionally, the formulation may further comprise decarboxylase enzyme inhibitors, such as carbidopa or benserazide. Typically, decarboxylase enzyme inhibitors may be present in an amount of about 10 mg to about 100 mg, and preferably in an amount of about 25 mg to about 50 mg per dose.

In one preferred embodiment, the amount per dose of levodopa is about 150 mg, the amount of carboxylase enzyme inhibitor is 50 mg, and the amount of methylphenidate is about 10 mg.

As discussed above, the timing of the administration of the individual ingredients of the composition of the invention is important to achieve the desired leveling of peaks and troughs of dopamine concentrations when treating Parkinson's disease. Generally, it is desirable to administer levodopa and optionally, a carboxylase enzyme inhibitor, prior to the administration of at least one dopamine transporter inhibitor. Alternatively, the levodopa, carboxylase enzyme inhibitor, and dopamine transporter inhibitor of the composition may be administered concurrently as a unit dose or co-administered as several doses. Each ingredient, however, may be formulated either as an immediate release formulation or sustained release formulation with or without a time delay. When administered, levodopa may be administered as an immediate release formulation or a sustained release delivery formulation wherein the levodopa is released over about 1 to about 4 hours. The carboxylase enzyme inhibitor may be dosed as an immediate drug delivery formulation or a sustained release delivery formulation wherein the carboxylase enzyme inhibitor is released over about 1 to about 4 hours. Typically, the dopamine transporter inhibitor is formulated as an immediate release formulation which releases after about a 2 hour to about 7 hour delay, and preferably after about a 3 to about 5 hour delay. Alternatively, the dopamine transporter inhibitor may be formulated as a sustained release delivery formulation which releases over one to six hours after about a 2 to about 7 hour delay.

For example, in one embodiment of the invention, the formulation comprises levodopa and at least one carboxylase enzyme inhibitor both in immediate release formulations and at least one dopamine transporter inhibitor in an immediate release formulation which releases after a 2 to 7 hour delay. In another embodiment of the invention, the levodopa and the carboxylase enzyme inhibitor are formulated in a sustained release delivery formulation, wherein the extended delivery comprises from about 1 to about 4 hours and the dopamine transporter inhibitor in an immediate release formulation after a 2 to 7 hour delay, preferably after a 3 to 5 hour delay.

In another embodiment, the formulation comprises levodopa and carboxylase enzyme inhibitors in immediate release formulations and the dopamine transporter inhibitor in a sustained release delivery formulation which releases over one to six hours after a 2 to 7 hour delay. In another embodiment, the formulation comprises levodopa and carboxylase enzyme inhibitors in a sustained release delivery, wherein the extended delivery comprises from about 1 to about 4 hours and the dopamine transporter inhibitor in a sustained release delivery formulation which releases over one to six hours after a 2 to 7 hour delay.

In another embodiment, the levodopa is formulated in a sustained release delivery formulation, wherein the sustained delivery comprises over about 1 to about 4 hours; the carboxylase enzyme inhibitor is formulated as an immediate release formulation; and at least one dopamine transporter inhibitor is formulated in an immediate release formulation which releases after a 2 to 7 hour delay. In another embodiment, the levodopa is formulated in a sustained release delivery formulation, wherein the sustained delivery comprises over about 1 to about 4 hours; the carboxylase enzyme inhibitor is formulated as an immediate release formulation; and at least one dopamine transporter inhibitor is formulated as a sustained release delivery formulation which releases over one to six hours after a 2 to 7 hour delay.

In a more preferred embodiment, the amount of levodopa is about 150 mg formulated in a sustained release delivery formulation released over 1 to 4 hours, the amount of carboxylase-enzyme inhibitor is 50 mg formulated in an immediate release formulation, and the amount of methylphenidate is about 10 mg formulated in a sustained release delivery formulation over 1 to 6 hours after a 2 to 7 hour delay. In another more preferred embodiment, the amount of levodopa is about 150 mg formulated in a sustained release delivery formulation released over 3 hours, the amount of carboxylase enzyme inhibitor is 50 mg formulated in an immediate release formulation or concurrent with levodopa, and the amount of methylphenidate is about 10 mg formulated in a sustained release delivery formulation over 3 hours after a 3 to 5 hour delay.

While the preceding embodiments are merely exemplary, one of ordinary skill in the art may easy determine other combinations of drug formulations, amounts, and administration as contemplated by the invention using the parameters disclosed herein.

The invention also encompasses a method of treating, preventing, or ameliorating Parkinson's disease in a mammal comprising administering to a mammal in need of such treatment a therapeutically effective amount of at least one of the formulations described herein. Methods of treating, preventing, or ameliorating Parkinson's disease in a mammal may comprise a dosage regime as described below.

One method for treating Parkinson's disease comprises administering levodopa and at least one dopamine transport inhibitor. The administration of the dopamine transport inhibitor may be delayed such that release coincides with the time the dopamine concentration level starts to decrease. The method may optionally include administering a decarboxylase enzyme inhibitor. A levodopa metabolic precursor like the levodopa ethyl ester of U.S. Pat. No. 5,840,756 may be substituted for levodopa in the various embodiments of the invention. In the method, typically, levodopa is administered in an amount from about 50 mg to about 300 mg, preferably from about 100 mg to about 200 mg and more preferably, levodopa is administered in an amount of about 150 mg.

The dopamine transporter inhibitors in the method include, but are not limited to, methylphenidate. The methylphenidate may be administered in an amount about 1 mg to about 60 mg, preferably from 1 mg to about 15 mg, more preferably, from about 5 mg to about 10 mg, and most preferably methylphenidate may be administered in an amount of about 10 mg per dose.

Optionally, the method of treating Parkinson's disease may further comprise administering decarboxylase enzyme inhibitors, such as carbidopa or benserazide. Typically, decarboxylase enzyme inhibitors may be administered in an amount of about 10 mg to about 100 mg, and preferably in an amount of about 50 mg per dose.

In one preferred embodiment, Parkinson's disease is treated by administering 150 mg of levodopa, 50 mg of carboxylase enzyme inhibitor, and 10 mg of methylphenidate. In another preferred embodiment, Parkinson's disease is treated by administering 100 mg of levodopa, 25 mg of carbidopa, and 10 mg of methylphenidate.

The levodopa, carboxylase enzyme inhibitor, and dopamine transporter inhibitor may be administered concurrently as a unit or co-administered. Each ingredient, however, may be administered either as an immediate release formulation or sustained release formulation with or without a time delay. Levodopa may be administered as an immediately release formulation or the levodopa may be released over a period of time of about 1 to about 4 hours. The carboxylase enzyme inhibitor may be administered immediately or over a sustained period of time of about 1 to about 4 hours. Typically, the dopamine transporter inhibitor is administered immediately after about a 2 hour to about 7 hour delay, preferably after about a 3 to about 5 hour delay. Alternatively, the dopamine transporter inhibitor may be administered over one to six hours after about a 2 to about 7 hour delay.

For example, in one embodiment of the invention, the treatment of Parkinson's disease comprises administering levodopa and at least one carboxylase enzyme inhibitor both in immediate release formulations and at least one dopamine transporter inhibitor in an immediate release formulation after a 2 to 7 hour delay. In another embodiment of the invention, the levodopa and the carboxylase enzyme inhibitor are administered as a sustained release delivery formulation, wherein the levodopa and the carboxylase enzyme inhibitor are delivered over a period of time of about 1 to about 4 hours and the dopamine transporter inhibitor is administered as an immediate release formulation after a 2 to 7 hour delay, preferably after a 3 to 5 hour delay.

In another embodiment, the treatment of Parkinson's disease comprises administering levodopa and carboxylase enzyme inhibitors as immediate release formulations and the dopamine transporter inhibitor is administered over a period of time of about one to six hours after about a two to seven hour delay. In another embodiment, the treatment of Parkinson's disease comprises administering levodopa and carboxylase enzyme inhibitors over about 1 to about 4 hours and the dopamine transporter inhibitor is administered over one to six hours after about a two to about seven hour delay.

In another embodiment, the treatment of Parkinson's disease comprises administering the levodopa over about 1 to about 4 hours; the carboxylase enzyme inhibitor immediately; and at least one dopamine transporter inhibitor immediately after a 2 to 7 hour delay. In another embodiment, the levodopa is administered over a period of time of about one to about four hours; the carboxylase enzyme inhibitor is administered immediately; and at least one dopamine transporter inhibitor is administered over one to six hours after about a two to about seven hour delay.

In a more preferred embodiment, the treatment of Parkinson's disease comprises administering about 150 mg of levodopa over 1 to 4 hours, 50 mg of carboxylase enzyme inhibitor immediately, and 10 mg of methylphenidate over 1 to 6 hours after a 2 to 7 hour delay. In another more preferred embodiment, 150 mg of levodopa is administered over 3 hours, 50 mg of carboxylase enzyme inhibitor is administered immediately which may be concurrently with levodopa, and 10 mg of methylphenidate is administered over 3 hours after a 3 to 5 hour delay.

To produce a dosage form that can release at least three drugs at two or three different rates and with preprogrammed delays, special dosage forms are used. In the embodiments of the invention wherein levodopa and carbidopa are designed to be released concomitantly the drugs may be formulated as bilayer tablets. Alternatively, levodopa and carbidopa may be formulated as a tablet within a tablet. The outer tablet may contain a levodopa carbidopa combination designed to be released together either as immediate release delivery patterns or as a sustained release delivery. The inner tablet may be formulated to release after the outer tablet has released the formulations. Optionally, the inner tablet may be formulated with a coating layer to help achieve the desired delay in time.

In a preferred embodiment, the drugs may be formulated into a core tablet held in a recessed fashion within an annular ring of drug material. Such a dosage form is described in U.S. patent application Ser. No. 10/419,536 entitled “Dosage Form with a Core Tablet of Active Ingredient Sheathed in a Compressed Angular Body of Powder or Granular Material, and Process and Tooling for Producing It,” filed on Apr. 21, 2003 and Ser. No. 10/379,338 entitled “Controlled Release Dosage Forms,” filed on Mar. 3, 2003 and are incorporated herein by reference. The outer annular ring is formulated with the levodopa and carboxylase enzyme inhibitor and formulated for either immediate release or sustained release delivery for the desired time. The inner core of the dosage form contains the dopamine transport inhibitor to be released after a delay which may be formulated for the desired release profile.

Another preferred embodiment of the invention uses the dosage form described in U.S. patent application Ser. No. 10/191,298 entitled “Drug Delivery System for Zero Order, Zero-Order Biphasic, Ascending or Descending Drug Delivery,” filed on Jul. 10, 2002, incorporated hereby by reference. The dopamine transport inhibitor may be formulated in the tablet mantle and release at the desired rate after a delay. The levodopa and carboxylase enzyme inhibitor may be formulated in the expanding plug and release at the desired rate upon entry into the stomach. Another embodiment of this invention may be achieved by formulating each of the drugs as pellets each with its own release profile and delay where applicable and delivering the mixture of the three pellets in a capsule using methods commonly known in the art.

Embodiments of the invention, wherein each drugs may be released at a different rate can be formulated as tri-layer tablets. Each layer of the tablet may have a distinct release profile. For example, a tablet within a tablet with an immediate release coating wherein the innermost tablet would be formulated with the dopamine transport inhibitor, the outer portion of the tablet formulated with levodopa, and the outer coating formulated with decarboxylase enzyme inhibitor. In another preferred embodiment, the drugs may be formulated into tablet held in a recessed fashion within an annular ring of drug material, as described above. The recessed core may be formulated as a delayed release of dopamine transport inhibitor at the desired release profile; the annular ring may be formulated to give the desired release profile of levodopa (immediate release and sustained release delivery); and an outermost coating layer may give an immediate release of decarboxylase enzyme inhibitor.

Another preferred embodiment could be achieved by using the delivery system described in U.S. patent application Ser. No. 10/191,298, wherein the dopamine transport inhibitor is formulated in the mantle and the expanding plug is a bilayer tablet. One layer of the bilayer tablet comprising levodopa formulated for sustained release delivery and the other layer comprising carboxylase enzyme inhibitor formulated to release at the desired rate. Yet another embodiment of the invention could be achieved by formulating each of the drugs as pellets each with its own release profile and delay where applicable and delivering the mixture of the three pellets in a capsule as commonly to one of ordinary skill in the art.

It should be noted that levodopa can be replaced in this invention with an appropriate prodrug of levodopa including any pharmaceutically suitable ester of levodopa such as, but not limited to, the methyl, ethyl, or propyl esters of levodopa, or combinations thereof.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### Enteric Coated Methylphenidate with an Annular Sheath of Levodopa and Carbidopa

An inner core in the form of a tablet was surrounded by an annular sheath as described below. The inner core was enteric coated methylphenidate and the annular sheath comprised levodopa and carbidopa.

The inner core was made first by making a granulate of methylphenidate, followed by forming a tablet and subsequently coating the tablet.

Methylphenidate Granulate:

Methylphenidate (150 grams), anhydrous lactose (420 grams), and hydroxypropylcellulose (Klucel LF™, 30 grams) were mixed in a Diosna P 1/6 high shear granulator at 380 rpm for 5 minutes. Purified water (60 grams) was added over the next minute while continuing to granulate at 380 rpm. The granulate was then massed for a further 10 seconds at the same speed. The formed granulate was dried for 30 minutes in a Diosna Mini Lab fluidized bed drier to less than 2% volatiles at an inlet temperature of 50° C. and a fan setpoint of 40%. The volatile content was tested at 105° C. using a Sartorious MA 30 LOD tester. The yield of dry granulate was 586.9 gram (98.4%). The dried granulate was next milled using an Erweka mill with a screen of 0.8 mm. The yield of the milled granulate was 583.5 grams (99.4%).

Tableting Mixture:

The milled, dry, methylphenidate granulate (502.5 grams), was mixed in the dry state with MicroceLac® 100 USP (178.6 grams), and hydroxypropylmethylcellulose (Methocel K15M™, 193.6 grams) in a 5 liter V mixer for 5 minutes. Magnesium stearate NF/EP (5.3 grams) was added and the V mixer operated for a further half a minute. The yield of the dry mix of powders was 875.2 grams.

Tablet Formation:

The dry mix powder was pressed into tablets on a Kilian RTS 20 tablet press using 5 mm flat faced punches. The tablets weighed an average of 71.8 mg (design 70±3.5 mg), had a hardness of 4 Kp (design 3-6 Kp) and a tablet thickness of 2.65 mm (design 2.4-2.7 mm). The weight of the tablets produced was 676.9 grams.

Enteric Coating:

Purified water (522 grams) was placed in a mixing vessel. Talc (19.2 grams), and triethylcitrate (38.4 grams) were added and the mixture was stirred for 15 minutes with a magnetic stirrer. Eudragit L-30 D55™ (639.6 grams) was added and the mixture stirred gently. The coating mixture was passed through a 150μ screen and then continually mixed gently.

Methylphenidate core tablets (676.9 grams) were placed in the drum of a Hi coater perforated pan coater and heated to 30° C. to 32° C. while the drum was turning at 7 rpm. The coating mixture was sprayed onto the tablets in the perforated pan coater turning at 12 rpm with the tablet bed maintained at 30° C. to 32° C. with the inlet air temperature set at 44° C. until an average of 8 mg per tablet of enteric coat had been added to the tablets. The tablets were air dried in the drum for five minutes after the spraying was halted and subsequently dried on an aluminum tray in a drying oven set at 40° C. for 24 hours. The yield of enteric coated tablets was 729.3 grams.

The annular sheath was made of a carbidopa and levodopa granulate as an outer mantle to the methlyphenidate tablet.

Carbidopa/Levodopa Granulate:

Carbidopa (191.7 grams), levodopa (708.3 grams), and polyvinylpyrollidone (Povidone K-30™, 100 grams) were added to a Diosna P1/6 high shear granulator and mixed for 5 minutes at 260 rpm. Over the next minute ethanol (95%, 120 grams) was added as a granulating solvent while the mass was being mixed at 260 rpm. The mixture was then massed at 520 rpm for 45 seconds. The wet granulate was then milled through a 2.5 mm screen in an Erweka mill and subsequently dried for 35 minutes in a Diosna Mini Lab fluidized bed drier to less than 2.5% volatiles at an inlet temperature of 50° C. and a fan setpoint of 55%. The volatile content was tested at 105° C. using a Sartorious MA 30 LOD tester. The yield of dry granulate was 851.8 gram (85.2%). The dry granulate was milled once again in a Quadro-Comil through an 1143μ screen to yield 820.2 grams of dried, milled granulate.

Tableting Mixture:

The milled, dry, carbidopa/levodopa granulate (612 grams) was placed in a 5 liter V mixer. MicroceLac® 100 USP (427.5 grams), polyethylene oxide (Polyox WSR-N-750™, 300 grams) and polyvinylpyrollidone (Povidone K-30™, 150 grams) were added and mixed in the V mixer for 5 minutes. Magnesium stearate NF/EP (10.5 grams) was added and the V mixer operated for a further half a minute. The yield of the dry mix of powders was 1493.5 grams.

Annular Sheath Tablet Formation:

The enteric coated methylphenidate inner cores were added to the tablet feeder and the carbidopa/levodopa tableting mixture was added to the powder feeder of a Manesty LP39 press using the special spring loaded core rod tooling for making the annular sheathed tablets. The lower punch was flat beveled of 11 mm diameter and an inner hole (for the core rod) of 5.5 mm diameter. The upper punch was flat beveled of 11 mm diameter with a protrusion that was 1.2 mm tall and 5.5 mm diameter with slight tapering. The final tablets so formed weighed an average of 526.9 mg (design 530±26 mg), had a hardness of 4.4 Kp (design 3-8 Kp) and a tablet thickness of 5.9 mm (design 5.8-6.0 mm). The weight of the tablets produced was 810.2 grams. Each tablet contained 130 mg levodopa and 35 mg carbidopa in the outer annular sheath and 10 mg methylphenidate in the enteric coated inner core.

### Example 2

### Drug Profile of the Tablet of Example 1

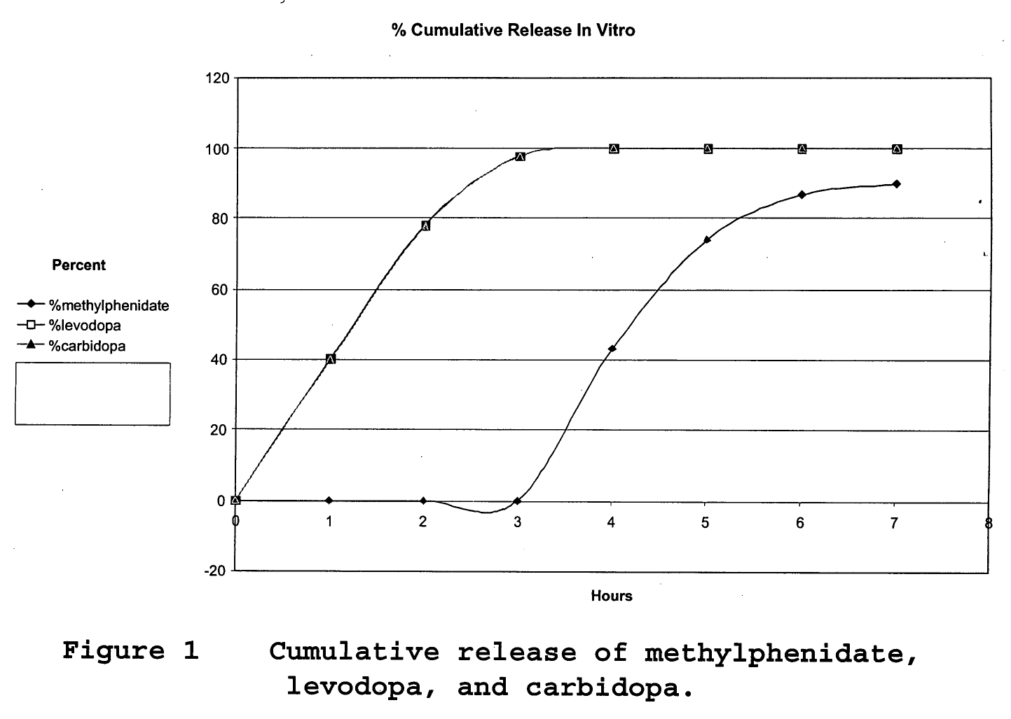

The tablets of Example 1 were tested to determine the drug release profile. The drug release was tested in an USP Apparatus II in 900 ml 0.1N HCl at 37° C. and 50 rpm for 3 hours and then pH=6.8 phosphate buffer for an additional 4 hours. At one hour intervals, the concentrations of methylphenidate, levodopa and carbidopa were measured by HPLC analysis. The results were summarized in Table 1 and illustrated in FIG. 1 for the drug release profile of the tablet of Example 1. The data demonstrated that the enteric coat prevented the methylphenidate from being released for the three hours that the system was in the acidic buffer. During the first three hours the majority of the levodopa (97.8%) and carbidopa (97.7%) was released. In contrast, none of the methlyphenidate was released over the first three hours. However, once the tablet was transferred to a neutral buffer, the methylphenidate was released over the following four hours to achieve a maximum release of 89.9% over the 7 hours of the study. See, Table 1.

TABLE 1
Cumulative release of methylphenidate, levodopa and carbidopa

| Time (hours) | Methylphenidate (%) | Levodopa (%) | Carbidopa (%) |
|---|---|---|---|
| 1 | 0 | 40.3 | 40.0 |
| 2 | 0 | 78.1 | 77.8 |
| 3 | 0 | 97.8 | 97.7 |
| 4 | 43.3 | n.m. | n.m. |
| 5 | 74.0 | n.m. | n.m. |
| 6 | 86.7 | n.m. | n.m. |
| 7 | 89.9 | n.m. | n.m. |

n.m. = not measured

### Example 3

### In Vivo Drug Release Study

A single-center, randomized, open-label, 2-treatment, 2 sequence, 2 period, cross-over pharmacokinetic study was carried out to determine the in vivo drug release profile of the tablets. The study was designed and carried out as follows.

“Day 1” was designated as the day in which the drug treatment was administered. In each study, two days prior to Day 1, designated “Day (−2)” and “Day (−1),” respectively, an oral pre-treatment regimen of 50 mg (2×25 mg) carbidopa (Lodosyn®, 25 mg; Merck & Co., Inc.) was administered 3 times a day. Each subject received a total dose of 150 mg/day of carbidopa during the 2 days prior to Day 1, the study drug administration. The pre-treatment administrations were ambulatory.

Two treatments were administered in the study, the single test formulation of the invention of three separate drugs combined into one tablet (levodopa, carbidopa, and methylphenidate) and a control consisting of two different tablets administered at different times. One tablet of the control contained levodopa/carbidopa administered at 0 hour, and the second tablet contained methylphenidate administered 3 hours thereafter. On Day 1 of each study period, after having fasted for at least 10 hours, the subjects received one of the treatments according to a randomization code.

The administered treatments are summarized as follows:

Treatment 1:

1×130 mg levodopa, 35 mg carbidopa, and 10 mg methylphenidate tablet in a tablet form as described in Example 1.

Treatment 2:

(a) 1×100 mg levodopa+25 mg carbidopa, administered as Sinemet-CR®, Merck & Co., Inc.; and
(b) 1×10 mg methylphenidate, administered as Ritalin®, Novartis. which was administered 3 hours following dosing of Sinemet-CR®.

The administered dose of levodopa selected for the trial was comparable to the low dose Sinemet-CR® as well as equivalent to the levodopa infusion dose (2 mg/kg) that was administered to the patients in a combination levodopa/methylphenidate study. See, Camicioli R et al., “Methylphenidate increases the motor effects of L-Dopa in Parkinson's disease: a pilot study,” Clinical Neuropharmacology, 24 (4), 208-213 (2001). The amount of carbidopa administered in the test formulation was approximately 25% that of levodopa, to maintain the recommended 1:4 ratio of carbidopa/levodopa. See, Sinemet-CR description, PHYSICIAN'S DESK REFERENCE, pp. 1111-1113 (57^{th} Ed., 2003).

The Test Tablet and Sinemet-CR® were swallowed whole with water (240 ml). The subjects assigned to Treatment 2 received a Ritalin® tablet 3 hours after receiving the Sinemet-CR® tablet with additional water (200 ml). Subjects assigned to Treatment 1 were required to drink additional water (200 ml) 3 hours after receiving the Test Tablet. After the first drug administration, the subjects remained in fasting for 5 hours. There was a washout period of at least 14 days between dosing periods of the Test Tablet or Sinemet-CR®, i.e. the second pre-treatment period with carbidopa commenced at least 12 days after administration of these medications.

The sampling method was performed as follows. For each of the study periods, 17 serial blood samples were collected per subject up to 12 hours following study drug administration. Samples were taken at time intervals of “0” hour (pre-dosing), 0.5, 1, 1.5, 2, 2.5, 3, 3.25, 3.5, 4, 4.5, 5, 6, 7, 8, 10, and 12 hours post-dose, for a total of 17 blood samples per study period (approximately 190 ml total per study session). Each blood sample (10 ml) measured the plasma concentration of levodopa and carbidopa as well as methylphenidate and its metabolite, ritanilic acid. The presence and amount of levodopa and carbidopa were determined using HPLC-ED or LC-MS-MS and for methylphenidate and ritanilic acid LC-MS-MS was used.

Tables 2 to 5 summarise the pharmacokinetic parameters for levodopa, carbidopa, methylphenidate, and ritanilic acid, respectively, for the Treatment 1 and Treatment 2. The tables provide average values for each parameter, AUC (h*ng/g), AUCinf (h*ng/g), t_{1/2}, Tₘₐₓ, and Cₘₐₓ.

TABLE 2
Pharmacokinetic parameters for Levodopa

| Treatment 1 | | | | | | Treatment 2 | | | | | |
|---|---|---|---|---|---|---|---|---|---|---|---|
| Vol./ | AUC | AUCinf | | Tₘₐₓ | Cₘₐₓ | Vol./ | AUC | AUCinf | | Tₘₐₓ | Cₘₐₓ |
| sess. | (h * ng/g) | (h * ng/g) | t_{1/2} | (h) | (ng/g) | sess. | (h * ng/g) | (h * ng/g) | t_{1/2} | (h) | (ng/g) |
| 1-1 | 3039.4 | 3039.4 | 1.3 | 1.0 | 1361.4 | 1-2 | 2006.0 | 2006.0 | 1.3 | 2.0 | 569.0 |
| 2-2 | 4142.2 | 4234.5 | 1.7 | 3.5 | 3063.6 | 2-1 | 2293.7 | 2293.7 | 1.6 | 3.0 | 619.9 |
| 3-1 | 3769.7 | 3769.7 | 1.6 | 2.0 | 1146.1 | 3-2 | 2770.7 | 2770.7 | 1.4 | 1.5 | 930.7 |
| 4-2 | 2244.9 | 2244.9 | 1.0 | 1.5 | 1023.2 | 4-1 | 1763.5 | 1763.5 | 1.2 | 1.5 | 904.4 |
| 5-2 | 1600.6 | 1600.6 | 1.4 | 1.0 | 816.8 | 5-1 | 1012.6 | 1012.6 | | 1.0 | 461.0 |
| 6-2 | 3161.5 | 3161.5 | 1.4 | 2.0 | 2788.4 | 6-1 | 2025.1 | 2025.1 | 0.9 | 2.5 | 1263.5 |
| 7-1 | 2216.2 | 2216.2 | 1.7 | 2.5 | 1134.4 | 7-2 | 1553.8 | 1553.8 | 1.5 | 2.0 | 449.0 |
| 8-1 | 1904.5 | 1904.5 | 1.4 | 1.0 | 781.3 | 8-2 | 1890.8 | 1890.8 | 1.0 | 0.5 | 767.1 |
| 9-1 | 1489.4 | 1489.4 | 1.3 | 0.5 | 455.7 | 9-2 | 2110.6 | 2110.6 | 1.2 | 2.0 | 787.4 |
| 10-2 | 2134.9 | 2134.9 | 1.6 | 4.0 | 571.0 | 10-1 | 2927.7 | 2927.7 | | 1.5 | 1030.0 |
| 11-1 | 3960.8 | 3960.8 | 1.8 | 1.5 | 2201.4 | 11-2 | 2684.7 | 2684.7 | 2.0 | 2.5 | 985.6 |
| 12-2 | 2857.2 | 2857.2 | 3.7 | 1.5 | 1221.8 | 12-1 | 2131.8 | 2131.8 | 1.2 | 2.5 | 721.5 |
| Avg. | 2710.1 | 2717.8 | 1.7 | 1.9 | 1380.4 | | 2097.6 | 2097.6 | 1.3 | 1.9 | 790.8 |
| Geom | 2567.1 | 2571.8 | 1.6 | 1.6 | 1174.8 | | 2026.3 | 2026.3 | 1.3 | 1.7 | 755.1 |
| Stdev | 916 | 930 | 0.7 | 1.1 | 850 | | 537 | 537 | 0.3 | 0.7 | 245 |
| Stderr | 264.5 | 268.3 | 0.2 | 0.3 | 245.3 | | 154.9 | 154.9 | 0.1 | 0.2 | 70.7 |

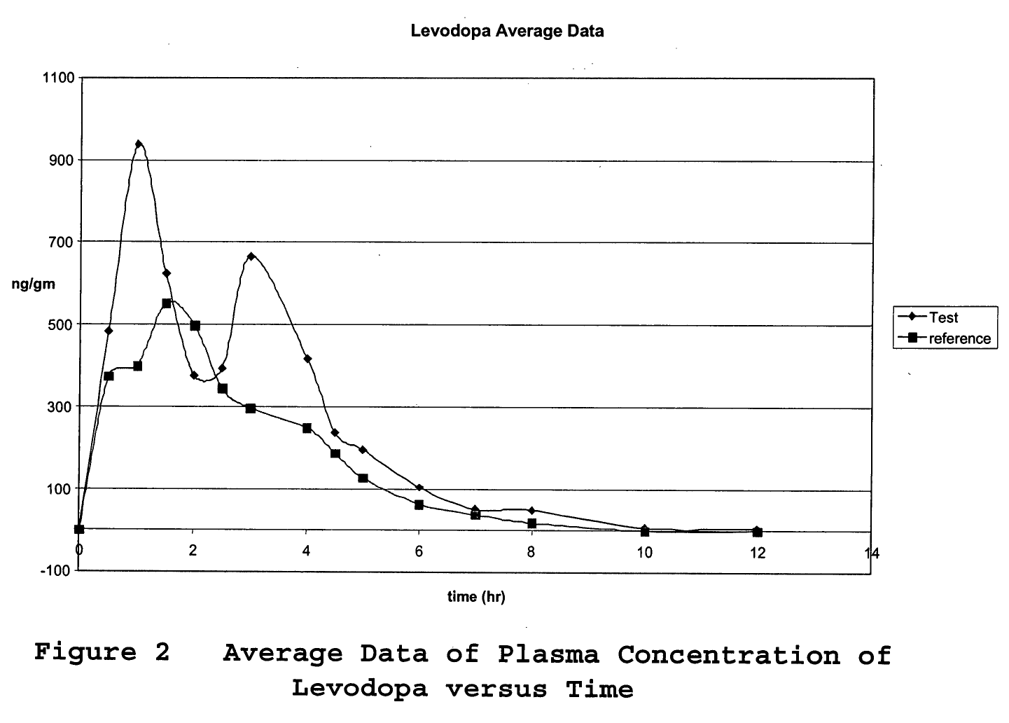

The data in Table 2 showed that the average AUC obtained for levodopa in Treatment 1 (tablet of the invention) was 2710 (h*ng/g) for a dose of 130 mg while for Treatment 2 (the reference treatment) was 2098 (h*ng/g) for a dose of 100 mg. Normalizing to 100 mg gave an AUC of 2085 (h*ng/g) for Treatment 1 compared to 2098 (h*ng/g) for Treatment 2. The bioavailability of levodopa from the two formulations was the same (99%) for equivalent doses. The average Tmax measured for levodopa in each of the treatments was at 1.9 hours. The average Cmax measured for Treatment 1 was 1380 (ng/g) for a dose of 130 mg which normalized to 1062 (ng/g) for a dose of 100 mg. Treatment 2 gave a Cmax of 791 (ng/g) for a dose of 100 mg. The terminal elimination half life was slightly longer for Treatment 1 than for Treatment 2. FIG. 2 illustrates the average graph of the concentration of levodopa in the blood versus time. Both treatments delivered drug for about 4 hours after which the elimination of the drug is the dominant feature of the graph.

TABLE 3
Pharmacokinetic Parameters for Carbidopa

| Treatment 1 | | | | | | Treatment 2 | | | | | |
|---|---|---|---|---|---|---|---|---|---|---|---|
| Vol./ | AUC | AUCinf | | Tₘₐₓ | Cₘₐₓ | Vol./ | AUC | AUCinf | | Tₘₐₓ | Cₘₐₓ |
| sess. | (h * ng/g) | (h * ng/g) | t_{1/2} | (h) | (ng/g) | sess. | (h * ng/g) | (h * ng/g) | t_{1/2} | (h) | (ng/g) |
| 1-1 | 702.7 | 702.7 | 1.7 | 4.5 | 140.2 | 1-2 | 119.6 | | | 3.3 | 43.7 |
| 2-2 | 407.5 | 407.5 | 2.0 | 4.5 | 107.0 | 2-1 | 287.8 | 287.8 | 2.0 | 3.3 | 102.0 |
| 3-1 | 406.5 | 406.5 | 2.6 | 4.5 | 83.8 | 3-2 | 383.6 | 383.6 | 3.2 | 3.3 | 144.3 |
| 4-2 | 488.2 | 488.2 | 2.1 | 3.0 | 146.1 | 4-1 | 297.3 | 297.3 | 3.2 | 3.0 | 79.6 |
| 5-2 | 15.9 | | | 1.5 | 31.9 | 5-1 | 0.0 | | | | 0.0 |
| 6-2 | 224.3 | 224.3 | 2.7 | 4.0 | 70.6 | 6-1 | 165.5 | 165.5 | 2.0 | 3.5 | 60.8 |
| 7-1 | 74.8 | | | 4.0 | 40.5 | 7-2 | 321.1 | 321.1 | 2.4 | 3.0 | 107.1 |
| 8-1 | 306.8 | 306.8 | 4.9 | 3.0 | 63.0 | 8-2 | 0.0 | | | | 0.0 |
| 9-1 | 0.0 | | | | | 9-2 | 423.2 | 423.2 | 1.5 | 2.5 | 121.4 |
| 10-2 | 403.9 | 403.9 | 1.7 | 4.5 | 122.7 | 10-1 | 349.4 | 349.4 | 2.4 | 2.5 | 110.7 |
| 11-1 | 603.2 | 603.2 | 1.7 | 4.5 | 125.2 | 11-2 | 506.4 | 506.4 | 1.9 | 3.5 | 130.9 |
| 12-2 | 451.9 | 451.9 | 2.2 | 2.0 | 112.3 | 12-1 | 339.9 | 339.9 | 2.0 | 4.0 | 87.8 |
| Avg. | 340.5 | 443.9 | 2.4 | 3.5 | 94.8 | | 266.1 | 341.6 | 2.3 | 3.2 | 82.4 |
| Geom | | 422.7 | 2.3 | 3.4 | 85.6 | | | 328.4 | 2.2 | 3.1 | |
| Stdev | 224 | 144 | 1.0 | 1.1 | 39 | | 161 | 95 | 0.6 | 0.5 | 48 |
| Stderr | 64.8 | 41.6 | 0.3 | 0.3 | 11.4 | | 46.6 | 27.4 | 0.2 | 0.1 | 13.8 |

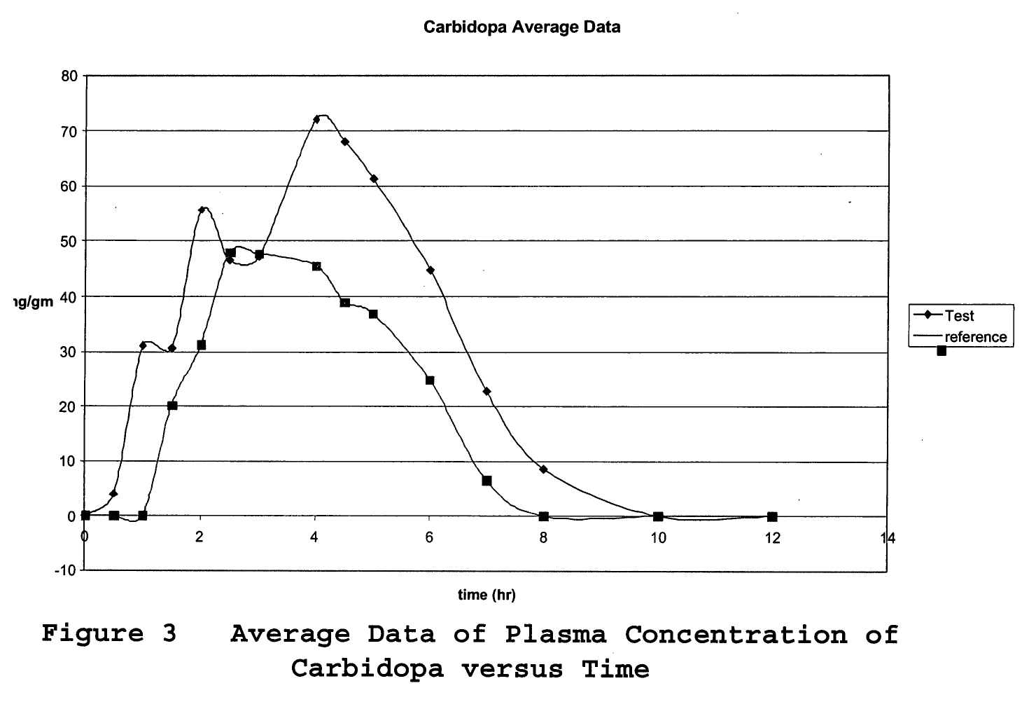

The data in Table 3 showed that the average AUC for carbidopa obtained for the Treatment 1 (tablet of the invention) was 340.5 (h*ng/g) for a 35 mg dose which normalized to 243 (h*ng/g) for a 25 mg dose. Treatment 2 (reference treatment) gave an average AUC of 266.1 (h*ng/g). The relative bioavailability of the two treatments for equivalent doses was similar (91%). The average Tmax measured was essentially the same for the two treatments, i.e., 3.5 and 3.2 hours for Treatment 1 and Treatment 2, respectively. The average elimination half life obtained was also very similar for the two treatments, 2.4 and 2.3 hours, respectively. The average Cmax measured for Treatment 1 was 94.8 (ng/g) for a 35 mg dose which was about 67.7 (ng/g) for a dose of 25 mg. Treatment 2 showed a higher Cmax with a value of 82.4 (ng/g). FIG. 3 illustrated the average graph of the concentration of levodopa in the blood versus time. Both treatments delivered drug for about 5-6 hours after which the elimination of the drug is the dominant feature of the graph. The profiles were similar in form.

TABLE 4
Pharmacokinetic Parameters for Methylphenidate

| Treatment 1 | | | | | | Treatment 2 | | | | | |
|---|---|---|---|---|---|---|---|---|---|---|---|
| Vol./ | AUC | AUCinf | | Tₘₐₓ | Cₘₐₓ | Vol./ | AUC | AUCinf | | Tₘₐₓ | Cₘₐₓ |
| sess. | (h * ng/g) | (h * pg/g) | t_{1/2} | (h) | (pg/g) | sess. | (h * pg/g) | (h * pg/g) | t_{1/2} | (h) | (pg/g) |
| 1-1 | 23769.3 | 26292.1 | 2.4 | 3.5 | 4188.6 | 1-2 | 26343.2 | 30305.5 | 2.9 | 4.0 | 6538.0 |
| 2-2 | 17465.5 | 25380.8 | 3.9 | 6.0 | 4315.9 | 2-1 | 17444.4 | 20659.2 | 2.6 | 6.0 | 4315.2 |
| 3-1 | 20875.4 | 26629.1 | 3.3 | 6.0 | 4314.8 | 3-2 | 25504.1 | 29341.8 | 2.8 | 4.5 | 5890.1 |
| 4-2 | 16931.2 | 20103.4 | 3.2 | 5.0 | 2650.6 | 4-1 | 19049.1 | 21933.2 | 2.8 | 5.0 | 4116.4 |
| 5-2 | 15609.8 | 18409.7 | 3.6 | 4.5 | 2395.4 | 5-1 | 16960.0 | 20259.8 | 3.2 | 4.0 | 3628.9 |
| 6-2 | 11093.1 | 12466.1 | 2.7 | 4.5 | 1913.9 | 6-1 | 11357.3 | 13059.8 | 3.0 | 4.5 | 2826.8 |
| 7-1 | 34833.7 | 47815.5 | 3.7 | 6.0 | 8281.7 | 7-2 | 31413.1 | 41718.3 | 3.8 | 5.0 | 6276.1 |
| 8-1 | 17789.9 | 20961.4 | 3.1 | 4.5 | 2769.9 | 8-2 | 21160.5 | 25504.3 | 2.5 | 4.5 | 5499.2 |
| 9-1 | 15036.6 | 18556.6 | 3.3 | 6.0 | 2592.4 | 9-2 | 17072.3 | 21493.7 | 3.6 | 4.5 | 4413.8 |
| 10-2 | 15768.9 | 20524.2 | 3.9 | 6.0 | 2526.8 | 10-1 | 16704.1 | 19319.7 | 2.7 | 4.5 | 3778.1 |
| 11-1 | 20403.8 | 24970.7 | 3.3 | 6.0 | 3636.5 | 11-2 | 22813.7 | 27409.9 | 3.3 | 4.5 | 5504.6 |
| 12-2 | 18697.6 | 24205.9 | 3.5 | 6.0 | 3625.1 | 12-1 | 16834.4 | 18946.9 | 2.8 | 4.5 | 4667.6 |
| Avg. | 19022.9 | 23859.6 | 3.3 | 5.3 | 3600.9 | | 20221.4 | 24162.7 | 3.0 | 4.6 | 4787.9 |
| Geom | 18314.7 | 22719.7 | 3.3 | 5.3 | 3332.7 | | 19557.1 | 23211.7 | 3.0 | 4.6 | 4655.9 |
| Stdev | 5934 | 8594 | 0.5 | 0.9 | 1688 | | 5480 | 7377 | 0.4 | 0.5 | 1150 |
| Stderr | 1712.9 | 2480.9 | 0.1 | 0.3 | 487.3 | | 1581.8 | 2129.6 | 0.1 | 0.2 | 322.0 |

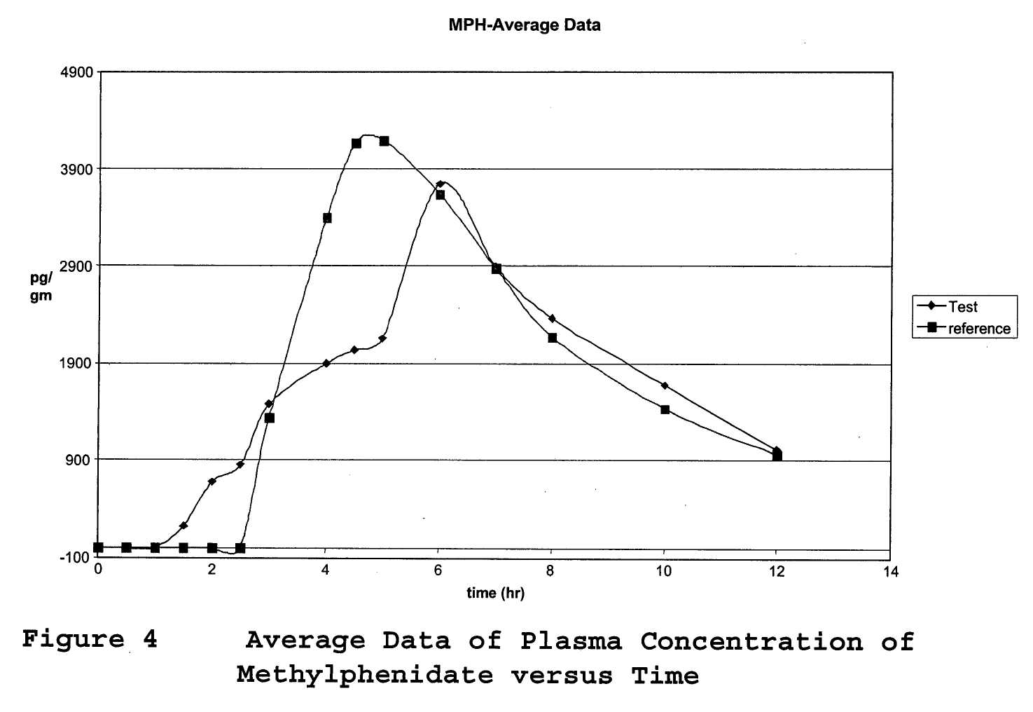

The data in Table 4 showed the data obtained for Treatment 1 (tablet of the invention) and Treatment 2 (reference treatment) for methylphenidate. In Treatment 1, 10 mg methylphenidate was in the same dosage form as the levodopa and carbidopa, was designed to give a delay after the Tmax of the levodopa, and to give a controlled release profile over 3 to 4 hours. In Treatment 2, the methylphenidate was an immediate release 10 mg tablet, dosed separately, three hours after levodopa and carbidopa dosing. The average AUC measured for Treatment 1 was 19023 (h*pg/g) while Treatment 2 had an average AUC of 20221 (h*pg/g). The relative bioavailability of Treatment 1 was 94%. The average Tmax measured for Treatment 2 was 4.6 hours from levodopa/carbidopa dosing or 1.6 hours after dosing of the immediate release tablet of methylphenidate. The average Tmax measured for Treatment 1 was 5.3 hours. The average Cmax measured for Treatment 1 was only 71% that of the average Cmax measured for Treatment 2, as expected for the comparison of a slow release versus an immediate release product. FIG. 4 illustrated the average graph for the concentration of methylphenidate in the blood versus time for the two treatments. Treatment 2 showed no methylphenidate in the first three hours since the drug was not yet dosed. The drug release in Treatment 2 was essentially complete after about 1.5 hours. Treatment 1 showed little drug release for the first two hours and then controlled release of the drug over the next 4-5 hours.

TABLE 5
Pharmacokinetic Parameters for Ritanilic Acid

| Treatment 1 | | | | | | Treatment 2 | | | | | |
|---|---|---|---|---|---|---|---|---|---|---|---|
| Vol./ | AUC | AUCinf | | Tₘₐₓ | Cₘₐₓ | Vol./ | AUC | AUCinf | | Tₘₐₓ | Cₘₐₓ |
| sess. | (h * ng/g) | (h * pg/g) | t_{1/2} | (h) | (pg/g) | sess. | (h * pg/g) | (h * pg/g) | t_{1/2} | (h) | (pg/g) |
| 1-1 | 794.2 | 1175.4 | 5.3 | 6.0 | 124.3 | 1-2 | 845.3 | 1202.6 | 4.9 | 5.0 | 168.9 |
| 2-2 | 510.3 | 1060.4 | 6.8 | 7.0 | 96.0 | 2-1 | 624.2 | 911.7 | 4.4 | 6.0 | 130.0 |
| 3-1 | 741.8 | 1189.6 | 5.8 | 6.0 | 158.0 | 3-2 | 874.7 | 1171.1 | 4.2 | 4.5 | 177.7 |
| 4-2 | 759.5 | 1028.3 | 4.3 | 4.5 | 131.4 | 4-1 | 767.4 | 1022.3 | 4.2 | 5.0 | 168.4 |
| 5-2 | 464.1 | 647.4 | 4.9 | 6.0 | 64.8 | 5-1 | 493.0 | 736.7 | 5.7 | 4.0 | 109.9 |
| 6-2 | 582.2 | 762.1 | 3.9 | 6.0 | 98.5 | 6-1 | 625.4 | 848.3 | 4.8 | 5.0 | 145.6 |
| 7-1 | 484.0 | 803.4 | 5.5 | 6.0 | 100.6 | 7-2 | 510.1 | 673.7 | 3.8 | 5.0 | 112.1 |
| 8-1 | 728.3 | 992.8 | 4.8 | 4.5 | 110.8 | 8-2 | 676.5 | 984.8 | 5.3 | 4.5 | 159.4 |
| 9-1 | 644.8 | 916.9 | 4.6 | 6.0 | 104.7 | 9-2 | 747.6 | 1051.2 | 4.8 | 4.5 | 171.6 |
| 10-2 | 648.7 | 931.2 | 4.3 | 6.0 | 108.5 | 10-1 | 754.5 | 1127.2 | 5.6 | 4.5 | 173.6 |
| 11-1 | 675.8 | 991.5 | 4.9 | 6.0 | 116.1 | 11-2 | 737.5 | 1097.5 | 5.1 | 4.5 | 131.1 |
| 12-2 | 684.1 | 998.6 | 4.4 | 6.0 | 130.6 | 12-1 | 770.4 | 1094.8 | 5.2 | 4.5 | 177.2 |
| Avg. | 643.1 | 958.1 | 5.0 | 5.3 | 112.0 | | 702.2 | 993.5 | 4.8 | 4.8 | 152.1 |
| Geom | 633.8 | 945.0 | 4.9 | 5.8 | 109.7 | | 692.1 | 979.0 | 4.8 | 4.7 | 150.0 |
| Stdev | 111 | 160 | 0.8 | 0.7 | 23 | | 120 | 169 | 0.6 | 0.5 | 25 |
| Stderr | 32.0 | 46.2 | 0.2 | 0.2 | 6.7 | | 34.7 | 48.8 | 0.2 | 0.1 | 7.3 |

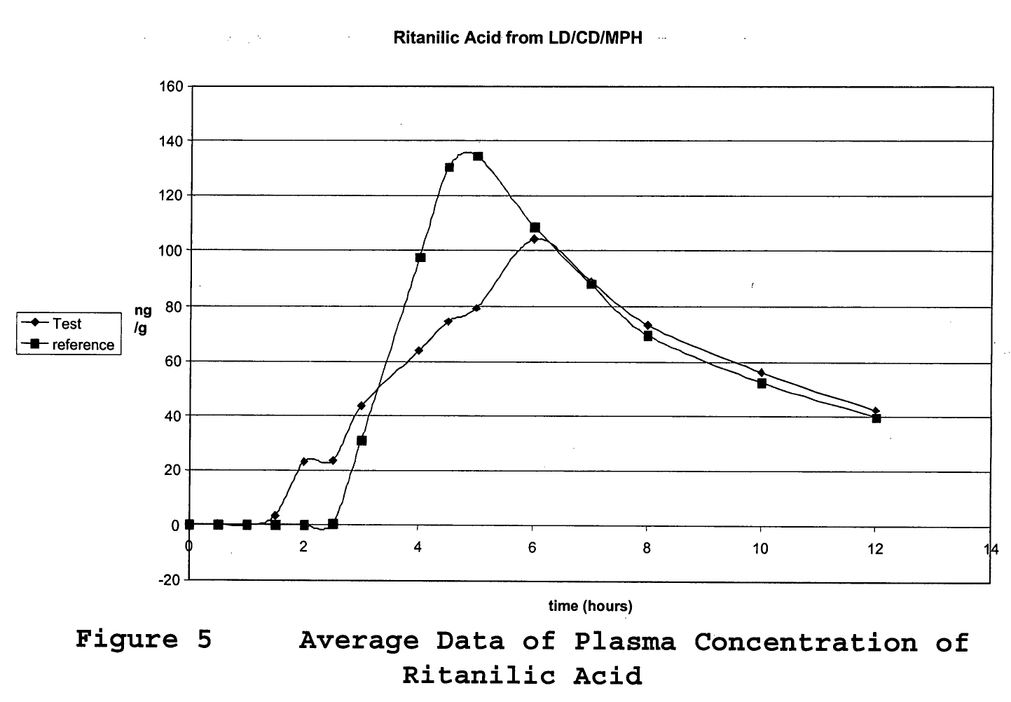

The data in Table 5 showed the data obtained for Treatment 1 (tablet of the invention) and Treatment 2 (reference treatment) for ritanilic acid, a metabolite of methylphenidate. The average AUC measured for Treatment 1 was 643.1 (h*ng/g) while Treatment 2 had an average AUC of 702.2 (h*ng/g). The relative bioavailability of Treatment 1 was therefore ˜92%. Since the metabolite showed significant concentrations at the last measured time point the AUC extrapolated to infinity may be a more accurate measurement for the relative bioavailability of this metabolite. The AUC_{inf} value for Treatment 1 was 958.1 (h*ng/g) while for Treatment 2 the value was 993.5 (h*ng/g), a relative bioavailability of 96%. The average Tₘₐₓ measured for Treatment 2 arm was 4.8 hours from levodopa/carbidopa dosing or 1.8 hours after dosing the immediate release tablet of methylphenidate. The average Tₘₐₓ measured for Treatment 1 was 5.3 hours, both values tracking the values for the parent drug. The average Cmax measured for the Treatment 1 was only 74% that of the average Cmax measured for Treatment 2 arm, as one would expect for the comparison of a slow release versus an immediate release product and again very similar to that found in the parent drug. The elimination half life for the ritanilic acid was about 5 hours in both treatments compared to about 3 hours for in the parent methylphenidate. FIG. 5 illustrates the average graph for the concentration of ritanilic acid in the blood versus time for the two treatment arms. The form of the graph is very similar to that of the parent drug except for the slower elimination profile.

1. A composition for the treatment of Parkinson's disease comprising:
a therapeutically effective amount of levodopa or a metabolic precursor thereof; and
at least one dopamine transport inhibitor in sufficient amount to decrease dopamine elimination, wherein the dopamine transport inhibitor is administered to avoid dyskinesia.

2. The composition according to claim 1, wherein the dopamine transport inhibitor is methylphenidate.

3. The composition according to claim 1, wherein the dopamine transport inhibitor is present in an amount of about 3 mg to about 60 mg.

4. The composition according to claim 1, wherein the levodopa or metabolic precursor thereof is present in an amount of about 50 mg to about 300 mg.

5. The composition according to claim 1, further comprising at least one carboxylase enzyme inhibitor.

6. The composition according to claim 5, wherein the carboxylase enzyme inhibitor is carbidopa, benserazide, or a combination thereof.

7. The composition according to claim 5, wherein the carboxylase enzyme inhibitor is present in an amount of about 10 mg to about 100 mg.

8. A pharmaceutical composition for treating, preventing, or ameliorating Parkinson's disease comprising:
an immediate release or sustained release delivery formulation of levodopa or a metabolic precursor thereof; and
a formulation of at least one dopamine transport inhibitor wherein the dopamine transport inhibitor is released immediately after a delay about 2 hours to about 7 hours.

9. The pharmaceutical composition according to claim 8, further comprising a decarboxylase enzyme inhibitor in an immediate release formulation or a sustained release delivery formulation.

10. The pharmaceutical composition according to claim 8 or 9, wherein the sustained release formulation is released from about 1 hour to about 4 hours.

11. The pharmaceutical composition according to claim 8, wherein the formulation is shaped into a bilayer tablet or sheathed tablet.

12. A pharmaceutical composition for treating, preventing, or ameliorating Parkinson's disease comprising:
an immediate release or sustained release delivery formulation of levodopa or a metabolic precursor thereof; and
a sustained release delivery formulation of at least one dopamine transport inhibitor wherein the dopamine transport inhibitor is released over a period of time of about 1 hour to about 6 hours after a delay of about 2 hour to about 7 hours.

13. The pharmaceutical composition according to claim 12, further comprising a decarboxylase enzyme inhibitor in an immediate release or a sustained release delivery formulation.

14. The pharmaceutical composition according to claim 8 or 13, wherein the sustained release formulation is released from about 1 hour to about 4 hours.

15. A method of treating Parkinson's disease comprising:
administering a therapeutically effective amount of levodopa or a metabolic precursor thereof; and
at least one dopamine transport inhibitor in sufficient amount to decrease dopamine elimination, wherein the dopamine transport inhibitor is administered to avoid dyskinesia.

16. The method according to claim 15, wherein the dopamine transport inhibitor is methylphenidate.

17. The method according to claim 15, wherein the dopamine transport inhibitor is administered in an amount of about 3 mg to about 60 mg.

18. The method according to claim 15, wherein the levodopa or metabolic precursor thereof is administered in an amount of about 50 mg to about 300 mg.

19. The method according to claim 15, further comprising administering at least one carboxylase enzyme inhibitor.

20. The method according to claim 19, wherein the carboxylase enzyme inhibitor is carbidopa, benserazide, or a combination thereof.

21. The method according to claim 19, wherein the carboxylase enzyme inhibitor is administered in an amount of about 10 mg to about 100 mg.
